(19) United States
(12) Patent Application Publication
(10) Pub. No.: US 2005/0042297 A1
(43) Pub. Date: Feb. 24, 2005

(54) Granules obtained by drying an emulsion comprising a polymer and a monosaccharide or a monosaccharide derivative

(76) Inventor: Melissa Manuszak, Paris (FR)

Correspondence Address: Jean-Louis Seugnet;Intellectual Property Dept, Rhodia Inc, 259 Prospect Plains Road, CN 7500, Cranbury, NJ, 08512-7500, US

(21) Appl. No.: 10/499,576

(22) PCT Filed: Dec. 18, 2002
(86) PCT No.: PCT/FR02/04427

(30) Foreign Application Priority Data
Dec. 27, 2001 (FR) 01/16941

(51) Int. Cl. A61K 9/14

(52) U.S. Cl. 424/489

(57) ABSTRACT

The invention concerns granules obtainable by drying an emulsion comprising: at least an active hydrophobic material; at least a polyakoxylated non ionic surfactant; at least a water soluble or water dispersible compound selected among polymers obtained from at least a monocarboxylic, polycarboxylic or carboxylic anhydride aliphatic, cyclic or aromatic ethylenically unsaturated monomer and from at least ethylenically unsaturated hydrocarbon monomer; at least a monosaccharide having 4 to 6 carbon atoms, its corresponding derivatives. The inventive granules can be used for detergency, skin and/or hair treatment, in pharmaceutics.

A subject matter of the present invention is granules obtained by drying an emulsion comprising a hydrophobic active material, more particularly an emulsion having a volatile component, and at least one monosaccharide or one reduced monosaccharide derivative.

Formulations in the form of emulsions are employed in numerous fields of application and problems related to the stability on storage of these emulsions, both with the regard to the stability of the emulsion itself (creaming, flocculation, ripening and coalescence) and also with regard to the stability of the active material (decomposition by hydrolysis, for example), can be encountered with such formulations.

In order to solve these problems, formulations in the form of powders or granules obtained from emulsions incorporating compounds capable of giving, after removal of the water, a solid matrix have been developed.

For example, there exist formulations obtained by drying emulsions comprising starch or starch derivatives. However, the use of this type of compound exhibits several disadvantages, in particular with regard to the drying. Furthermore, the dried products exhibit a degree of sensitivity to moisture and can become sticky during storage, resulting in agglomeration of the granules. Furthermore, the product can become yellowish and therefore less attractive to the user and, finally, it can have a discoloring effect on the clothes which it may come into contact with.

There also exist formulations obtained by drying emulsions comprising a polymeric compound, such as, for example, carboxylic acid/hydrocarbonaceous compound copolymers. One of the difficulties encountered with formulations of this type lies in the fact that a portion of the hydrophobic active material present in the emulsion, in particular if one or more of its components is volatile, can be removed during the drying.

The present invention makes it possible to include the techniques described above.

A subject-matter of the present invention is thus granules capable of being obtained by drying an emulsion comprising:
at least one hydrophobic active material,
at least one nonionic polyalkoxylated surfactant,
at least one water-soluble or water-dispersible compound chosen from polymers obtained from at least one aliphatic, cyclic or aromatic monocarboxylic acid, polycarboxylic acid or carboxylic anhydride monomer carrying an ethylenic unsaturation and from at least one hydrocarbonaceous monomer carrying at least one ethylenic unsaturation,
the emulsion additionally comprising at least one monosaccharide exhibiting 4 to 6 carbon atoms, or corresponding derivatives.

This is because it has been found, unexpectedly, that the presence of a compound of the type of the monosaccharides or derivatives in the emulsion before it is dried makes it possible to significantly reduce the amount of volatile hydrophobic active material removed during the drying.

A subject matter of the present invention is likewise the use of the granules in formulations employed in the fields of detergency, of the treatment of the skin and/or hair, or in the pharmaceutical industry.

However, other advantages and characteristics of the present invention will become more clearly apparent on reading the description which will follow.

As is indicated above, the emulsion comprises at least one hydrophobic active material.

More specifically, the active materials employed exist in the form of a liquid which is immiscible or very slightly miscible with water or in a form dissolved in an organic solvent, the combination being immiscible or very slightly miscible with water. The term “slightly miscible” is understood to mean active materials with a solubility in water not exceeding 10% by weight between 20° C. and the temperature of preparation of the emulsion.

Subsequently, in the account, the term “active material” will be understood as meaning either the pure active material or the active material dissolved in an organic solvent.

Preferably, the active material employed is in the form of a liquid at a temperature of between 20° C. and the temperature of preparation of the emulsion. However, it is not impossible for the active material to be a solid with a melting point of less than or equal to 100° C., more particularly of less than or equal to 80° C.

In addition, it is specified that the present invention is very particularly suitable for hydrophobic active materials, one of the components of which is volatile.

The term “volatile” denotes active materials which evaporate either at atmospheric pressure and at 20° C. and/or under the drying conditions employed for the preparation of the granules and/or the active materials which form an azeotrope with water under the drying conditions employed for the preparation of the granules.

Mention may be made, as suitable active materials in the context of the present invention, of flavorings, fragrances, essential oils or essences. Mention may be made, by way of examples, of oils and/or essences of mint, of spearmint, of peppermint, menthol, of vanilla, of cinnamon, of bay, of anise, of eucalyptus, of thyme, of sage, of cedar leaf, of nutmeg, of citrus (lemon, lime, grapefruit, orange), of fruits (apple, pear, peach, cherry, plum, strawberry, raspberry, apricot, pineapple, grape, and the like), alone or as mixtures. Use may also be made of compounds such as benzaldehyde, isoamyl acetate, ethyl butyrate, and the like.

Mention may also be made, among suitable hydrophobic active materials, of:
organic oils/fats/waxes of animal origin or of vegetable origin;
inorganic oils/waxes;
products resulting from the alcoholysis of the abovementioned oils;
mono-, di- and triglycerides;
saturated or unsaturated fatty acids comprising 10 to 40 carbon atoms; the esters of such acids and of alcohol comprising 1 to 6 carbon atoms;
saturated or unsaturated monoalcohols comprising 8 to 40 carbon atoms; these compounds being used alone or as a mixture.

Mention may be made, as organic oils/fats/waxes of animal origin, inter alia, of sperm oil, whale oil, seal oil, shark oil, cod liver oil, pig or sheep fats (tallows), perhydrosqualene, beeswax, alone or as a mixture.

Mention may be made, as examples of organic oils/fats/waxes of vegetable origin, inter alia, of rapeseed oil, sunflower oil, ground nut oil, olive oil, walnut oil, corn oil, soybean oil, avocado oil, linseed oil, hemp oil, grape seed oil, coconut oil, palm oil, cottonseed oil, palm kernel oil, babassu oil, jojoba oil, sesame oil, castor oil, macadamia oil, sweet almond oil, carnauba wax, karite butter, cocoa butter, peanut butter, alone or as a mixture.

Mention may be made, as regards mineral oils/waxes, inter alia, of naphthenic, paraffin (liquid petrolatum) or isoparaffin oils, paraffin waxes, alone or as a mixture.

The products resulting from the alcoholysis of the abovementioned oils can also be used.

As regards fatty acids, the latter, which may or may not be saturated, comprise 10 to 40 carbon atoms, more particularly 18 to 40 carbon atoms, and can comprise one or more conjugated or nonconjugated ethylenic unsaturations. It should be noted that said acids can comprise one or more hydroxyl groups.

Mention may be made, as examples of saturated fatty acids, of palmitic acid, stearic acid or behenic acid.

Mention may be made, as examples of unsaturated fatty acids, of myristoleic acid, palmitoleic acid, oleic acid, erucic acid, linoleic acid, linolenic acid, arachidonic acid, ricinoleic acid, and their mixtures.

Mention may be made, as regards esters of fatty acids, of the esters of the acids listed above for which the part deriving from the alcohol comprises 1 to 6 carbon atoms, such as the methyl, ethyl, propyl or isopropyl esters, and the like.

Silicone oils, belonging in particular to the family of dimethicones, silicone resins and silicone gums can also be employed.

Lipophilic vitamins, such as vitamin A and its derivatives, vitamin B2, pantothenic acid, vitamin D and vitamin E; UV absorbing agents, such as aminobenzoate derivatives of PABA and PARA type, salicylates or cinnamates.

Antiaging agents, such as ceramides, phospholipids, cholesterol, sterols and their mixtures.

The active materials which have just been mentioned can be used in the fields of cosmetics, detergency or the pharmaceutical industry. It is specified that they are given by way of illustration and are not to be regarded as exhaustive.

Finally, the active materials which have just been described can be used, alone or as mixtures, as solvent of one or more other active materials.

The total amount of active material present in the emulsion is such that it represents, in the dried granule, from 1 to 60% by weight, preferably between 5 and 50% by weight.

The emulsion additionally comprises at least one nonionic polyalkoxylated surfactant.

More particularly, these surfactants are chosen from the following, alone or as mixtures:
polyalkoxylated fatty alcohols;
polyalkoxylated mono-, di- and triglycerides;
polyalkoxylated fatty acid esters;
polyalkoxylated sorbitan esters;
polyalkoxylated fatty acid amides;
polyalkoxylated fatty amines;
polyalkoxylated amidoamines;
polyalkoxylated di(1-phenylethyl)phenols;
polyalkoxylated tri(1-phenylethyl)phenols;
polyalkoxylated alkylphenols;
polyalkoxylated polysiloxanes;
products resulting from the condensation of ethylene oxide or of propylene oxide with ethylenediamine;
polyalkoxylated terpene hydrocarbons;
polyalkoxylated alkylpolyglycosides, alone or in mixtures.

The term “(poly)alkoxylated” denotes ethoxylated or propoxylated units, or their combinations. Preferably, the surfactants comprise ethoxylated or ethoxylated/propoxylated units.

The number of ethoxylated (EO) and/or propoxylated (PO) units in these surfactants usually varies from 1 to 100, more particularly from 1 to 50.

The EO or EO/PO fatty alcohols generally comprise from 6 to 22 carbon atoms, the EO and PO units being excluded from these numbers. Preferably, these units are ethoxylated (EO) units.

The EO or EO/PO, preferably EO, mono-, di- and triglycerides are more particularly mono-, di- and triglycerides of vegetable or animal origin which are optionally hydrogenated, as, for example, such as linseed oil, soybean oil, castor oil, rapeseed oil, and the like).

The EO or EO/PO fatty acid esters generally comprise, for the acid part, from 6 to 22 carbon atoms, the EO and PO units being excluded from these numbers, and are preferably ethoxylated (EO).

The EO or EO/PO, preferably EO, sorbitan esters are fatty acid esters of cyclized sorbitol where the fatty acid comprises from 10 to 20 carbon atoms, such as lauric acid, stearic acid or oleic acid.

The term “EO or EO/PO mono-, di- and triglycerides” is targeted, in the present invention, both at the products obtained by alkoxylation of a mono-, di- and triglyceride by ethylene oxide and/or by propylene oxide and are those obtained by transesterification of a mono-, di- and triglyceride by a polyethylene glycol and/or polypropylene glycol.

Likewise, the term “EO or EO/PO fatty acid ester” includes both the products obtained by alkoxylation of a fatty acid by ethylene oxide and/or propylene oxide and those obtained by transesterification of a fatty acid by a polyethylene glycol and/or polypropylene glycol.

The EO or EO/OP fatty amines and amides generally have from 6 to 22 carbon atoms, the EO and PO units being excluded from these numbers, and are preferably ethoxylated (EO).

The EO or EO/PO amidoamines generally have from 2 to 22 carbon atoms for the hydrocarbonaceous units, the EO and PO units being excluded from these numbers, and are preferably ethoxylated (EO).

The EO or EO/PO alkylphenols generally have one or two linear or branched alkyl groups having 4 to 12 carbon atoms. Mention may be made, by way of examples, of in particular the octyl, nonyl or dodecyl groups.

The preferably ethoxylated (EO) polysiloxanes are more particularly linear or branched polyalkylsiloxanes comprising 2 to 10 silicon atoms in which the alkyl groups are preferably methyl radicals.

The suitable, preferably EO or EO/PO, terpene hydrocarbons are in particular those derived from α- or from β-pinenes. They have been disclosed in International Application WO 96/01245.

The alkylpolyglycosides can be obtained by condensation of glucose with primary fatty alcohols exhibiting a C₄-C₂₀ alkyl group, and a mean number of glucose units of the order of 0.5 to 3 per mole of alkylpolyglycoside.

Preferably, the surfactant is chosen from polyalkoxylated, hydrogenated or nonhydrogenated, mono-, di- and triglycerides.

According to an alternative form of the invention, the nonionic polyalkoxylated surfactant is used in combination with at least one cosurfactant chosen from saturated or unsaturated fatty acids, for example such as those comprising 8 to 24 carbon atoms, and their polyalkoxylated (EO or EO/PO) derivatives. Said acids can be in an acid or salified form, the counterion being chosen from alkali metal (sodium, potassium, lithium) or unsubstituted or substituted ammonium (methyl-, dimethyl-, trimethyl-, or tetramethylammonium, dimethylpiperidinium, and the like) cations or cations derived from an alkanolamine (monoethanolamine, diethanolamine, triethanolamine, and the like).

In the case where the emulsion comprises at least one cosurfactant, the content of this compound, expressed as dry weight, represents 20 to 50% as dry weight of nonionic polyalkoxylated surfactant.

Furthermore, according to an advantageous embodiment of the invention, the total content in the emulsion of nonionic polyalkoxylated surfactant and optionally of cosurfactant, expressed as dry weight, is less than or equal to 20% by weight, preferably of between 0.5 and 10% by weight.

The emulsion furthermore comprises at least one water-soluble or water-dispersible compound.

This compound is chosen from the polymers obtained from at least one aliphatic, cyclic or aromatic monocarboxylic acid, polycarboxylic acid or carboxylic anhydride monomer carrying an ethylenic unsaturation and from at least one hydrocarbonaceous monomer carrying at least one ethylenic unsaturation.

As regards more particularly the acid monomer, the latter corresponds to the following formula:
(R¹)HC═C(R²)COOM
in which formula:
R¹ represents a hydrogen atom, a —COOM group or a —(CH₂)ₙ—COOM group in which n is between 1 and 4, or C₁-C₄ alkyl radical; R² represents a hydrogen atom, a (CH₂)ₘ—COOM group in which m is between 1 and 4, or a C₁-C₄ alkyl radical; M represents a hydrogen atom, an alkali metal (such as, for example, sodium, potassium, lithium or magnesium) or an ammonium group of NR₄⁺ type with R, which are identical or different, representing an alkyl radical which is or is not substituted by an oxygen atom; said monomer being used alone or as a mixture, or in the form of macromonomers of one or more of them. It is recalled that the term “macromonomer” denotes a macromolecule carrying one or more polymerizable functional groups.

Preferably, said acid monomer is such that the R¹ radical represents a hydrogen atom, a —COOM or —(CH₂)—COOM group or a methyl radical, and the R² radical represents a hydrogen atom, a —CH₂—COOM group or a methyl radical.

According to a highly advantageous embodiment of the present invention, the acid monomer is chosen from acrylic, methacrylic, citraconic, maleic, fumaric, itaconic or crotonic acids or anhydrides, alone or as a mixture, their salts, or in the form of macromonomers of one or more of them.

As regards the hydrocarbonaceous monomer, the latter corresponds more especially to the following formula:
(R²) (R²)—C═CH₂ (II)
in which formula the R² radicals, which are identical or different, represent a hydrogen atom, a linear or branched, cyclic, or cycloaliphatic, C₁-C₁₀ aliphatic radical optionally carrying an ethylenic unsaturation; or an aromatic radical optionally substituted by at least one C₁-C₁₀ alkyl radical optionally carrying an ethylenic unsaturation. Said monomers can, here again, be used alone or as a mixture, or in the form of macromonomers of one or more of them.

Thus, the abovementioned hydrocarbonaceous monomer can advantageously be chosen from ethylene, propylene, 1-butene, isobutylene, n−1-pentene, 2-methyl-1-butene, n-1-hexene, 2-methyl-1-pentene, 4-methyl-1-pentene, 2-ethyl-1-butene, diisobutylene, 2-methyl-3,3-dimethyl-1-pentene, styrene, α-methylstyrene, vinyltoluene, butadiene, chloroprene, isoprene or their mixtures, and the macromonomers deriving from such monomers.

Preferably, the R² radicals, which are identical or different, represent a hydrogen atom or a saturated, linear or branched, cyclic, or cycloaliphatic, C₁-C₁₀ aliphatic radical.

The monomers and their respective proportions are chosen so that the resulting copolymer is water-soluble or water-dispersible.

The term “water-soluble or water-dispersible” denotes the polymers which do not precipitate or which do not sediment when they are in solution/dispersion in the aqueous phase, optionally with the surfactant, under the conditions of preparation of the emulsion (concentration); the temperature being between 20 and 30° C.

Preferably, the distribution of the monomers in the copolymer is random. Of course, it would not be departing from the scope of the invention to use block copolymers.

By way of indication, the weight-average molar mass of the abovementioned polymers is more particularly less than or equal to 50 000 g/mol, advantageously less than or equal to 20 000 g/mol (absolute value determined by steric exclusion chromatography coupled to a MALLS (Multi-Angle Laser Light Scattering) analysis).

The compounds employed in the emulsion are copolymers well known to a person skilled in the art. They are obtained conventionally by carrying out a radical polymerization or a polymerization of anionic type.

Mention may be made, as copolymers of this type, of those of maleic anhydride and of an olefin. Mention may be made, among compounds of this type, of Geropon T36, sold by Rhodia Chimie.

In accordance with an alternative form of the invention, the ratio by weight of water-soluble or water-dispersible compound to hydrophobic active material, expressed as dry weight in the emulsion, is between 4/1 and 1/2, preferably between 4/1 and 1/1.5.

According to an essential characteristic of the invention, the emulsion from which the granules are obtained comprises at least one monosaccharide exhibiting 4 to 6 carbon atoms, or its derivatives. Said derivatives are more particularly reduced monosaccharides, and their corresponding disaccharides or trisaccharides. The monosaccharides can exist in an open or cyclic form. It is specified that they are not polymeric compounds.

Mention may be made, among the compounds of this type, of ketoses or aldoses, such as erythrose, threose (C₄), lyxose, xylose, arabinose, ribose (C₅), gulose, glucose, fructose, mannose, sorbose or sucrose (C₆), alone or as mixtures.

As regards the reduced derivatives of the preceding compounds, mention may be made of sorbitol, mannitol or adonitol, alone or as mixtures. Derivatives of the 1,4-anhydro-d-sorbitol type and mannitan are also suitable.

Finally, disaccharides may be suitable for the implementation of the invention, with, for example, sucrose, lactose or maltose, and trisaccharides, such as raffinose, alone or as a mixture.

In accordance with a first possibility for implementing the invention, the monosaccharide or derivative is used as substitute for the water-soluble or water-dispersible compound. In this scenario, the ratio by weight, expressed as weight of dry matter, of monosaccharide or derivative to water-soluble or water-dispersible compound is preferably less than or equal to 20/80; preferably, said ratio is between 1/99 and less than 20/80.

In accordance with another embodiment of the invention, the monosaccharide is used in combination with the water-soluble or water-dispersible compound. According to this specific embodiment, the content of monosaccharide, expressed as dry weight, represents 1 to 20% by weight of the emulsion, preferably from 1 to 5% by weight.

According to one embodiment of the invention, the emulsion from which the granules are obtained additionally comprises at least one complexing agent comprising at least one of the elements from Groups IIa, IVa, VIII, Ib and IIIb (according to the Periodic Table of the Elements which appeared in the Bulletin de la Société chimique de France, No. 1, January 1966).

More specifically, the complexing agent comprises at least one element chosen from calcium, magnesium, titanium, zirconium, copper, iron, aluminum, boron and their mixtures.

Depending on the nature of the elements, the complexing agents can create ionic interactions with the water-soluble or water-dispersible compound. This is, for example, the case with the ions of the borate, aluminate, titanate or titanyl sulfate type, and the like.

The complexing agents can likewise create interactions of the hydrogen bond type with the compound, such as, for example, the calcium, the zirconium, the iron, the cobalt or titanium, but also the oxides of the metals, such as, for example, of titanium or of zirconium, said oxides being in the form of nanometric particles.

The counterions of the complexing agents which have just been mentioned are preferably chosen from inorganic or organic ions which give entities which are soluble in aqueous media when they are combined with the complexer.

Mention may be made, by way of examples of inorganic anionic counterions, of halides, such as, for example, chlorides, carbonates, nitrates, sulfates, hydrosulfates, alkyl sulfates (for example comprising 1 to 6 carbon atoms), phosphates, citrates, formates or acetates.

Mention may in particular be made, as organic anionic counterions, of acetates or formates.

Mention may be made, by way of examples of cationic counterions, of alkali metals or ammonium ions of the NR₄⁺ type with R, which are identical or different, representing an alkyl radical which is or is not substituted by an oxygen atom.

More particularly, the molar ratio of number of metal atoms to number of hydroxyl and/or carboxyl functional groups of the compound is between 1/1 and 1/100.

The granules according to the invention are therefore capable of being obtained from an emulsion by carrying out the following stages:
an emulsion is prepared from an aqueous solution comprising the water-soluble or water-dispersible compound, the nonionic polyalkoxylated surfactant and optionally the cosurfactant, optionally at least one monosaccharide or derivative and the active material;
optionally at least one monosaccharide or derivative and optionally the complexing agent is/are added to the emulsion thus obtained;
the resulting mixture is dried.

According to a preferred embodiment of the invention, first a solution of the active material(s), optionally in a form dissolved in an appropriate solvent, is prepared. Secondly, an aqueous solution comprising the surfactant and the water-soluble or water-dispersible compound is prepared. According to a first embodiment of the invention, the monosaccharide or derivative is added to the aqueous solution. Then the aqueous solution is added to the solution of hydrophobic active material(s) with stirring.

The emulsion is prepared more particularly at a temperature of less than 100° C. and preferably between 20 and 60° C. and more preferably still at a temperature of between 20 and 40° C. It should be noted that the temperature at which the emulsion is prepared is such that the various ingredients are in a liquid form, so as to obtain an emulsion.

The mean particle size of the emulsion is generally between 0.1 and 10 μm, more particularly between 0.1 and 5 μm, preferably less than or equal to 1 μm. The mean size corresponds to the median diameter by volume (d₅₀), which represents the diameter of the particle equal to 50% of the cumulative distribution. It is measured using a Horiba particle sizer.

The amount of dry matter in the emulsion is generally between 10 and 60% by weight.

In the case where a complexing agent is used, it is introduced into the emulsion once the latter has been obtained. Advantageously, the completing agent is added in the form of an aqueous solution.

It should be noted that, according to a second embodiment of the invention, the monosaccharide or derivative is added in the form of an aqueous solution once the emulsion has been obtained.

The third stage of the preparation process according to the invention consists in drying the emulsion thus formulated in order to obtain granules.

The method employed to remove the water from the emulsion and to obtain granules can be carried out by any means known to a person skilled in the art.

This operation takes place in such a way that the various constituent components of the emulsion are subjected to temperatures below those at which they decompose.

According to a first embodiment of the invention, oven drying can be envisaged. Preferably, this drying takes place in a thin layer. More particularly, the temperature at which the drying is carried out is less than or equal to 100° C., preferably between 50 and 90° C.

According to another specific embodiment of the invention, “rapid” drying of the emulsion is carried out. Drying by atomization, in a fluidized bed, employing Duprat® drum dryers, or lyophilization (freezing/sublimation) are suitable in this respect.

Drying by atomization or in a fluidized bed can be carried out in the usual way in any known device, such as, for example, a spray tower, in which spraying can be carried out by a nozzle or a rotary disk in combination with a stream of hot gas. The inlet temperature of the hot gas (generally air) at the column top is preferably between 100 and 250° C. and the outlet temperature is preferably below the decomposition temperature of the constituent components of the granule obtained.

In the case of drying operations on the emulsion carried out using a Duprat® drum dryer or any means which makes it possible to rapidly obtain a dry film which is separated from the drying support by a scraping operation, for example, particles are obtained which can optionally be milled. If necessary, these particles can form the subject of a subsequent forming, such as an agglomeration stage, so as to obtain granules and/or tablets.

It is specified that it would not be departing from the scope of the invention to mix granules of different compositions.

As mentioned above, the invention also relates to the use of the granules in formulations employed in the fields of detergency, of the treatment of the skin and/or hair, or in the pharmaceutical industry. The granules can, for example, participate in formulations and/or in the form of powders, of agglomerates, of woven or nonwoven textile or paper surfaces, such as wipes, which are impregnated or coated, or in the form of tablets.

A concrete but nonlimiting example of the invention will now be presented.

### EXAMPLE

The composition, for 100 g of emulsion, is as follows:

NB: The amounts are expressed as dry weight.

| Product | Comp. | Invention |
|---|---|---|
| Geropon T36 (sold by Rhodia Geronazzo) | 17.39 g | 17.39 g |
| Maleic anhydride/olefin copolymer (as an | | |
| approximately 26% aqueous solution) | | |
| Fragrance | 20 g | 20 g |
| Alkanol OR40 (sold by Rhodia Chimie) | 5 g | 5 g |
| Ethoxylated castor oil | | |
| Neosorb 70/20 (sold by Roquette) | — | 5 g |
| Sorbitol as a 70% aqueous solution | | |

Preparation of the Granules:

First of all the aqueous phase is prepared by dissolution of the surfactant in the aqueous polymer solution and then the fragrance is added.

The combined mixture is stirred at 1000 rev/min for 10 minutes.

The emulsions are dried under air in a Niro Minor Technology atomizer by spraying using a disk rotating at 23 000 rev/min; the inlet temperature of the air is 105-110° C.; the outlet temperature of the air is 75-80° C. The emulsion flow rate is 3.15 kg/h.

A flowable powder is obtained at the outlet of the atomizer.

The water content of the powder, analyzed by Karl Fischer titration, is between 3.3 and 3.7.

Results:

The quantitative determination of the fragrance in the granules is carried out by head space coupled to gas chromatography (total evaporation technique) in water.
a) First of all the total amount of fragrance in the granules is determined:
The powder is milled and then diluted by half in water (w/weighed=0.2 to 0.8 g).
The sample is stirred for approximately 2 hours using a magnetic bar and a test sample of approximately 5 mg to 10 mg of this dispersion is withdrawn in a head space tube.
Calibration is carried out with a sample of fragrance (Beer-Lambert curve).
b) The content of fragrance removed corresponds to the difference between the initial content of fragrance and the total content of fragrance in the granules.

The analyses have shown that the loss of fragrance during the drying of the emulsion according to the invention is 13% by weight, whereas that observed during the drying of the emulsion according to the comparative example is 57% by weight.

1-12. (Canceled).

13. A granule made by the process of drying an emulsion comprising:
at least one hydrophobic active material,
at least one nonionic polyalkoxylated surfactant, and
at least one water-soluble or water-dispersible compound which is a polymer obtained from at least one aliphatic, cyclic or aromatic monocarboxylic acid, polycarboxylic acid or carboxylic anhydride monomer carrying an ethylenic unsaturation and from at least one hydrocarbonaceous monomer carrying at least one ethylenic unsaturation, said emulsion comprising at least one monosaccharide exhibiting 4 to 6 carbon atoms, or its derivatives.

14. The granule as claimed in claim 13, wherein the monosaccharide derivatives are reduced monosaccharides, from disaccharides or from trisaccharides.

15. The granule as claimed in claim 13, wherein the monosaccharide or derivative is used as substitute for the water-soluble or water-dispersible compound and with a ratio by weight, expressed as weight of dry matter, of monosaccharide to water-soluble or water-dispersible compound of less than or equal to 20/80.

16. The granule as claimed in claim 15, wherein the ratio is between 1/99 and less than 20/80.

17. The granule as claimed in claim 13, wherein the monosaccharide is in a content, expressed as dry weight, representing 1 to 20% by weight of the emulsion.

18. The granule as claimed in claim 17, wherein the content represents 1 to 5% by weight.

19. The granule as claimed in claim 13, presenting a ratio by weight, expressed as dry weight of compound in the emulsion, of water-soluble or water-dispersible compound to hydrophobic active material of between 4/1 and ½.

20. The granule as claimed in claim 19, wherein the ratio is between 4/1 and 1/1.5.

21. The granule as claimed in claim 13, wherein the emulsion further comprises at least one cosurfactant which is a saturated or unsaturated fatty acid.

22. The granule as claimed in claim 21, wherein the cosurfactant is in a content, expressed as dry weight, representing 20 to 50% as dry weight of nonionic polyalkoxylated surfactant.

23. The granule as claimed in claim 13, having a total content of nonionic polyalkoxylated surfactant and optionally of cosurfactant, expressed as dry weight in the emulsion, of less than or equal to 20% by weight.

24. The granule as claimed in claim 23, wherein the content is between 0.5 and 10% by weight.

25. The granule as claimed in claim 13, wherein the emulsion further comprises at least one crosslinking agent comprising at least one of the elements from Groups IIa, IVa, VIII, lb and IIIb.

26. The granule as claimed in claim 25, wherein the element of the crosslinking agent is calcium, magnesium, titanium, zirconium, copper, iron, aluminum or boron.

27. The granule as claimed in claim 25, having a molar ratio of number of metal atoms to number of carboxyl functional groups of the water-soluble or water-dispersible compound of between 1/1 and 1/100.

28. A formulation for detergency, the treatment of the skin or hair, or the pharmaceutical industry, comprising granules as defined in claim 13.
